(19) United States
(12) Patent Application Publication
(10) Pub. No.: US 2017/0275347 A1
(43) Pub. Date: Sep. 28, 2017

(54) METHODS FOR IDENTIFYING PATIENTS RESPONSIVE TO ANTI-PD-L1 ANTIBODY THERAPY

(71) Applicant: MedImmune Limited, Cambridge (GB)

(72) Inventors: Brandon HIGGS, Gaithersburg, MD (US); Jiaqi HUANG, Gaithersburg, MD (US); Wei ZHU, Gaithersburg, MD (US); Philip BROHAWN, Gaithersburg, MD (US); Katie Streicher, Gaithersburg, MD (US); Yihong YAO, Gaithersburg, MD (US)

(21) Appl. No.: 15/508,514

(22) PCT Filed: Sep. 4, 2015
(86) PCT No.: PCT/EP2015/070281

§ 371 Date: Mar. 3, 2017

Related U.S. Application Data
(60) Provisional application No. 62/066,576, filed on Oct. 21, 2014.
(60) Provisional application No. 62/046,420, filed on Sep. 5, 2014.

(51) Int. Cl. C07K 14/47; G01N 33/574; C12Q 1/68; C07K 14/52; C07K 16/28

(52) U.S. Cl. CPC C07K 14/4741; C07K 14/522; C07K 16/2827; C12Q 1/6886; G01N 33/57407; G01N 33/57423; C12Q 1/686; A61K 2039/505

(57) ABSTRACT

The disclosure provides methods for treating lung cancer (e.g., non-small cell lung cancer) with an anti-PD-L1 antibody in a patient identified using a polynucleotide or polypeptide marker of the disclosure: CXCL9, KRT8, TRIM29, and/or IFNgamma.

### BACKGROUND OF THE INVENTION

Lung cancer is among the most common forms of cancer and is the leading cause of cancer deaths among men and women. More people die of lung cancer annually than of colon, breast, and prostate cancers combined. Non-small cell lung cancer is the most common form of lung cancer. While the risk of acquiring lung cancer is higher among patients with a history of smoking, lung cancer also affects non-smokers. Improving survival of lung cancer patients remains difficult despite improved medical therapies. Most lung cancer is detected only in advanced stages when therapy options are limited. There is a growing recognition that lung cancer and other malignancies arise from a variety of pathogenic mechanisms. Methods of characterizing these malignancies at a molecular level is useful for stratifying patients, thereby quickly directing them to effective therapies. Improved methods for predicting the responsiveness of subjects having lung cancer, including NSCLC, are urgently required.

### SUMMARY OF THE INVENTION

The present invention provides methods for treating lung cancer (e.g., non-small cell lung cancer) with an anti-PD-L1 antibody in a patient identified using a polynucleotide or polypeptide marker of the invention (e.g., CXCL9, KRT8, TRIM29, and/or IFNgamma).

In one aspect, the invention generally provides a method of treatment involving administering an anti-PD-L1 antibody, or an antigen binding fragment thereof, to a patient identified as having a non-small cell lung cancer tumor that expresses one or more markers that are any one or more of CXCL9, KRT8, TRIM29, and IFNgamma. In one embodiment, the anti-PD-L1 antibody is MEDI4736.

In another aspect, the invention generally provides a method of treatment involving administering an anti-PD-L1 antibody, or an antigen binding fragment thereof, to a patient identified as having a head and neck cancer tumor that expresses one or more markers that are any one or more of CXCL9, KRT8, TRIM29, and IFNgamma. In one embodiment, the anti-PD-L1 antibody is MEDI4736.

In another aspect, the invention provides a method of treatment involving administering MEDI4736 or an antigen binding fragment thereof to a patient identified as having a non-small cell lung cancer tumor that expresses one or more markers that are any one or more of CXCL9, KRT8, TRIM29, and IFNgamma.

In another aspect, the invention provides a method of treatment involving administering MEDI4736 or an antigen binding fragment thereof to a patient identified as having a head and neck cancer tumor that expresses one or more markers that are any one or more of CXCL9, KRT8, TRIM29, and IFNgamma.

In yet another aspect, the invention provides a method of treatment involving administering MEDI4736 or an antigen binding fragment thereof to a patient identified as having a head and neck cancer tumor or a small cell lung cancer tumor that expresses CXCL9 and IFNgamma.

In another aspect, the invention provides a method of identifying a subject having non-small cell lung cancer or head and neck cancer responsive to anti-PD-L1 therapy, the method involving detecting an increase in the level of one or more markers that are any one or more of CXCL9, KRT8, TRIM29, and IFNgamma in a non-small cell lung cancer tumor or head and neck cancer tumore of the subject, relative to a reference, thereby identifying said non-small cell lung cancer as responsive to anti-PD-L1 therapy. In one embodiment, the method further involves detecting PD-L1 expression in the tumor. In other embodiments, the markers are detected by a method selected from real-time PCR, DNA microarray, immunostaining, ELISA, FACS, radioimmunoassay, immunoblot, Western blot, immunofluorescence, or immunoprecipitation.

In another aspect, the invention provides a set of primers and/or probes for characterizing non-small cell lung cancer (NSCLC) where the primers and/or probes hybridize to two or more polynucleotide markers that are any one or more of CXCL9, KRT8, TRIM29, and IFNgamma.

In another aspect, the invention provides a set of primers and/or probes for characterizing non-small cell lung cancer (NSCLC) where the primers and/or probes hybridize to three or more polynucleotide markers that are any one or more of CXCL9, KRT8, TRIM29, and IFNgamma. In one embodiment, the primers and/or probes hybridize to markers CXCL9, KRT8, TRIM29, and IFNgamma.

In another aspect, the invention provides a kit containing the primers and/or probles of any one of any previous aspect. In one embodiment the kit contains a reagent to measure the level of PD-L1.

In various embodiments of the above aspects or any other aspect of the invention herein, the patient is identified as responsive to MEDI4736. In various embodiments of the above aspects or any other aspect of the invention herein, the patient is further identified as having a tumor expressing PD-L1. In various embodiments of the above aspects or any other aspect of the invention herein, the tumor expresses CXCL9 and KRT8; CXCL9 and TRIM29; CXCL9, KRT8, TRIM29, and IFNgamma; or CXCL9, KRT8, TRIM29, IFNgamma, and PD-L1. In various embodiments of the above aspects, at least about 0.1, about 0.3, about 1, about 3, about 10, or about 15 mg/kg MEDI4736, or an antigen-binding fragment thereof, is administered In other embodiments, at least about 1 mg/kg, 3 mg/kg, 10 mg/kg, or 15 mg/kg MEDI4736, or an antigen-binding fragment thereof, is administered. In other embodiments, the administration is repeated about every 14 or 21 days. In other embodiments, at least two, three, four, or five doses is administered. In various embodiments of the above aspects or any other aspect of the invention herein, marker gene expression is detected in a Real-Time PCR assay. In various embodiments of the above aspects or any other aspect of the invention herein, marker polypeptide expression is detected using immunohistochemistry. In various embodiments of the above aspects or any other aspect of the invention herein, the tumor cells are formalin fixed and paraffin embedded. In various embodiments of the above aspects or any other aspect of the invention herein, the non-small cell lung cancer is squamous cell carcinoma, non-squamous cell carcinoma, adenocarcinoma, large cell carcinoma, adenosquamous carcinoma or sarcomatoid carcinoma.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham. The Harper Collins Dictionary of Biology (1991). As used herein, the following terms have the meanings ascribed to them below, unless specified otherwise.

By “Chemokine, CXC Motif, Ligand 9 (CXCL9) protein” is meant a polypeptide or fragment thereof having T-cell chemoattractant activity. The sequence of an exemplary CXCL9 polypeptide (Uniprot Accession No. Q07325) is provided below:

>sp|Q07325|CXCL9_HUMAN C-X-C motif chemokine 9
OS = Homo sapiens GN = CXCL9 PE = 1 SV = 1
MKKSGVLFLLGIILLVLIGVQGTPVVRKGRCSCISTNQGTIHLQSLK
DLKQFAPSPSCEKIEIIATLKNGVQTCLNPDSADVKELIKKWEKQVS
QKKKQKNGKKHQKKKVLKVRKSQRSRQKKTT

By “CXCL9 polynucleotide” is meant a polynucleotide encoding a CXCL9 protein. The sequence of an exemplary CXCL9 polynucleotide is provided at NCBI Accession No. NM_002416.1.

By “Keratin 8 (KRT8) polypeptide” is meant a structural protein or fragment thereof present in epithelial cells. An exemplary KRT8 amino acid sequence (Uniprot Accession No. P05787) is provided below:

sp|P05787|K2C8_HUMAN Keratin, type II
cytoskeletal 8 OS = Homo sapiens GN = KRT8
PE = 1 SV = 7
MSIRVTQKSYKVSTSGPRAFSSRSYTSGPGSRISSSSFSRVGSSNFR
GGLGGGYGGASGMGGITAVTVNQSLLSPLVLEVDPNIQAVRTQEKEQ
IKTLNNKFASFIDKVRFLEQQNKMLETKWSLLQQQKTARSNMDNMFE
SYINNLRRQLETLGQEKLKLEAELGNMQGLVEDFKNKYEDEINKRTE
MENEFVLIKKDVDEAYMNKVELESRLEGLTDEINFLRQLYEEEIREL
QSQISDTSVVLSMDNSRSLDMDSIIAEVKAQYEDIANRSRAEAESMY
QIKYEELQSLAGKHGDDLRRTKTEISEMNRNISRLQAEIEGLKGQRA
SLEAAIADAEQRGELAIKDANAKLSELEAALQRAKQDMARQLREYQE
LMNVKLALDIEIATYRKLLEGEESRLESGMQNMSIHTKTTSGYAGGL
SSAYGGLTSPGLSYSLGSSFGSGAGSSSFSRTSSSRAVVVKKIETRD
GKLVSESSDVLPK

By “KRT8 polynucleotide” is meant a nucleic acid molecule encoding a KRT8 polypeptide. The sequence of an exemplary KRT8 polynucleotide is provided at NCBI Accession No. NG_008402.1.

By “Tripartite Motif-Containing Protein 29 (”TRIM29″) protein” is meant a polypeptide or fragment thereof that physically associates with vimentin. The sequence of an exemplary TRIM29 polypeptide (Uniprot Accession No. Q14134) is provided below:

>sp|Q14134|TRI29_HUMAN Tripartite motif-
containing protein 29 OS = Homo sapiens
GN = TRIM29 PE = 1 SV = 2
MEAADASRSNGSSPEARDARSPSGPSGSLENGTKADGKDAKTTNGHG
GEAAEGKSLGSALKPGEGRSALFAGNEWRRPIIQFVESGDDKNSNYF
SMDSMEGKRSPYAGLQLGAAKKPPVTFAEKGELRKSIFSESRKPTVS
IMEPGETRRNSYPRADTGLFSRSKSGSEEVLCDSCIGNKQKAVKSCL
VCQASFCELHLKPHLEGAAFRDHQLLEPIRDFEARKCPVHGKTMELF
CQTDQTCICYLCMFQEHKNHSTVTVEEAKAEKETELSLQKEQLQLKI
IEIEDEAEKWQKEKDRIKSFTTNEKAILEQNFRDLVRDLEKQKEEVR
AALEQREQDAVDQVKVIMDALDERAKVLHEDKQTREQLHSISDSVLF
LQEFGALMSNYSLPPPLPTYHVLLEGEGLGQSLGNFKDDLLNVCMRH
VEKMCKADLSRNFIERNHMENGGDHRYVNNYTNSFGGEWSAPDTMKR
YSMYLTPKGGVRTSYQPSSPGRETKETTQKNENNLYGTKGNYTSRVW
EYSSSIQNSDNDLPVVQGSSSFSLKGYPSLMRSQSPKAQPQTWKSGK
QTMLSHYRPFYVNKGNGIGSNEAP

By “TRIM29 polynucleotide” is meant a polynucleotide encoding a TRIM29 polypeptide. The sequence of an exemplary TRIM29 polynucleotide is provided at NCBI Accession No. NM_012101.3.

By “Interferon gamma (IFNgamma) protein” is meant a polypeptide or fragment thereof having immunomodulatory activity. An exemplary IFNgamma amino acid sequence (Uniprot Accession No. P01579) is provided below:

>sp|P01579|IFNG_HUMAN Interferon gamma
OS = Homo sapiens GN = IFNG PE = 1 SV = 1
MKYTSYILAFQLCIVLGSLGCYCQDPYVKEAENLKKYFNAGHSDVAD
NGTLFLGILKNWKEESDRKIMQSQIVSFYFKLFKNFKDDQSIQKSVE
TIKEDMNVKFFNSNKKKRDDFEKLTNYSVTDLNVQRKAIHELIQVMA
ELSPAAKTGKRKRSQMLFRGRRASQ

By “IFNgamma polynucleotide” is meant a nucleic acid molecule encoding IFNgamma. The sequence of an exemplary IFNgamma polynucleotide is provided at NCBI Accession No. NM_000619.

By “anti-PD-L1 antibody” is meant an antibody or antigen binding fragment thereof that selectively binds a PD-L1 polypeptide. Exemplary anti-PD-L1 antibodies are described for example at U.S. Patent No. 8,779,108 and U.S. Patent Application Publication No. US2013034559, which is herein incorporated by reference. MEDI4736 is an exemplary PD-L1 antibody. Following treatment with MEDI4736, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≧50% relative to baseline. Confirmation can be obtained using a consecutive repeat assessment at least 4 weeks from the date of first documentation.

“Stable disease” (SD) indicates a decrease in tumor burden of 50% relative to baseline cannot be established and a 25% increase compared to nadir cannot be established.

By “PD-L1 polypeptide” is meant a polypeptide or fragment thereof having at least about 85%, 95% or 100% amino acid identity to NCBI Accession No. NP_001254635 and having PD-L1 and CD80 binding activity.

| PD-L1 polypeptide sequence | |
|---|---|
| NCBI ACCESSION NO. NP_001254635 | |
| 1 | mrifavfifm tywhllnapy nkinqrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

By “PD-L1 nucleic acid molecule” is meant a polynucleotide encoding a PD-L1 polypeptide. An exemplary PD-L1 nucleic acid molecule sequence is provided at NCBI Accession No. NM_001267706.

| PD-L1 nucleic acid sequence | |
|---|---|
| NCBI ACCESSION NO. NM_001267706 mRNA | |
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab')₂, Fv, scFv, Fd, dAb, and other antibody fragments that retain antigen-binding function, i.e., the ability to bind PD-L1 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab', F(ab')2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab')2 fragment in which the two arms of the antibody molecule remain linked and comprise two-antigen binding sites. The F(ab')2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab', single chain V region fragments (scFv), fusion polypeptides, and unconventional antibodies.

By “biologic sample” is meant any tissue, cell, fluid, or other material derived from an organism. In one embodiment, a biological sample is a tumor biopsy sample.

A “biomarker” or “marker” as used herein generally refers to a protein, nucleic acid molecule, clinical indicator, or other analyte that is associated with a disease. In one embodiment, a marker is differentially present in a biological sample obtained from a subject having a disease (e.g., lung cancer) relative to the level present in a control sample or reference.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Lung cancer includes small cell lung cancer (SCLC) and non-small cell lung cancer (NSCLC). There are three main subtypes of NSCLC: squamous cell carcinoma, adenocarcinoma, and large cell (undifferentiated) carcinoma. Other subtypes include adenosquamous carcinoma and sarcomatoid carcinoma. Head and Neck cancer comprises Laryngeal and Hypopharyngeal Cancer, Nasal Cavity and Paranasal Sinus Cancer, Nasopharyngeal Cancer, Oral and Oropharyngeal Cancer, and Salivary Gland Cancer.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reference” is meant a standard of comparison.

By “responsive” in the context of therapy is meant susceptible to treatment.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶M⁻¹, or more preferably higher than 10⁸M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 shows PD-L1 membrane expression in micrographs at baseline in non-small cell lung cancer (NSCLC) patients treated with anti-PD-L1 antibody (MEDI4736). The micrograph at left shows negligible PD-L1 staining. This patient did not respond to anti-PD-L1 antibody therapy and continued to show progressive disease (PD). In fact, there was a 43% tumor increase at 6 weeks (PD). The micrograph at right shows strong PD-L1 staining. This patient showed a partial response (PR) to anti-PD-L1 antibody therapy, displaying a 90% tumor decrease at 6 weeks (uPR).

FIG. 2 is a scatter plot showing a correlation between percent tumor size change from baseline and IHC M-score of PD-L1 defined as the percentage tumor cells with PD-L1 staining in NSCLC patients from CP1108 trial. Clinical response status was measured as best overall response (BOR) indicated by colors at (left) 6 weeks and (right) 12 weeks post dosing with anti-PD-L1. An immunohistochemistry membrane (IHC M) score greater than or equal to 25 is considered positive for PD-L1. In general, patients with an IHC M score greater than or equal to 25 responded to anti-PD-L1. Abbreviations: SD=stable disease; PR=partial responder; PD=progressive disease; ND=no clinical response status assigned.

FIG. 3 shows a heat map depicting the expression profiling of a panel of 125 candidate genes in NSCLC patients treated for 6 or 12 weeks with anti-PD-L1 antibody. Heatmap shows the scaled expression intensities across all NSCLC patient samples collected to date in CP1108 clinical trial. Red indicates high gene expression and blue indicates low gene expression. Black indicates no measurement taken for that patient/gene.

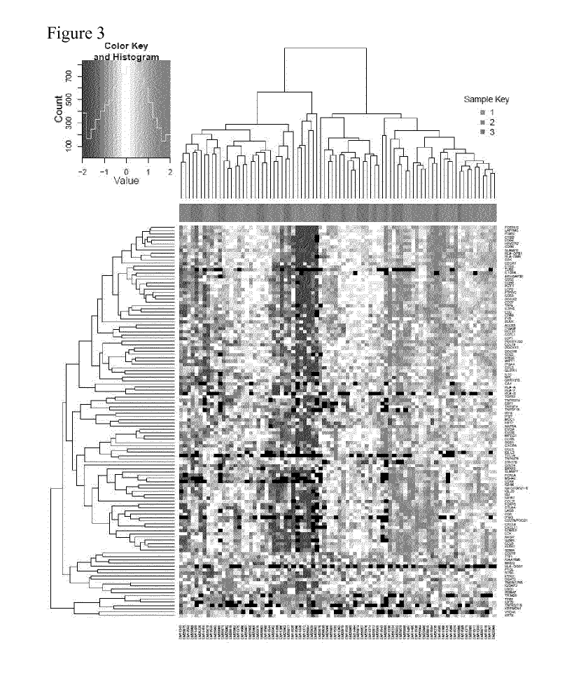

FIG. 4 is a scatter plot showing a correlation between percent tumor size change from baseline and baseline gene expression transformed to a final gene expression score. The relative gene expression (RGE) is defined as (20-Δcycle threshold, so that this score is positive overall and a high score represents high expression in the log2 scale. RGE values are provided for CXCL9 in NSCLC patients from the trial. Clinical response status was measured as best overall response (BOR) indicated by colors at (left) 6 weeks and (right) 12 weeks post dosing with anti-PD-L1. SD=stable disease; PR=partial responder; PD=progressive disease; ND=no clinical response status assigned.

FIG. 5 is a scatter plot showing a correlation between percent tumor size change from baseline and baseline gene expression transformed RGE values for KRT8 in NSCLC patients from trial. Clinical response status was measured as best overall response (BOR) indicated by colors at (left) 6 weeks and (right) 12 weeks post dosing with anti-PD-L1. SD=stable disease; PR=partial responder; PD=progressive disease; ND=no clinical response status assigned.

FIG. 6 is a scatter plot showing a correlation between percent tumor size change from baseline and baseline gene expression transformed RGE values for TRIM29 in NSCLC patients from trial. Clinical response status measured as best overall response (BOR) indicated by colors at (left) 6 weeks and (right) 12 weeks post dosing with anti-PD-L1. SD=stable disease; PR=partial responder; PD=progressive disease; ND=no clinical response status assigned.

FIG. 7 is a scatter plot showing a correlation between percent tumor size change from baseline and baseline gene expression transformed RGE values for IFNγ in NSCLC patients from the trial. Clinical response status measured as best overall response (BOR) indicated by colors at (left) 6 weeks and (right) 12 weeks post dosing with anti-PD-L1. SD=stable disease; PR=partial responder; PD=progressive disease; ND=no clinical response status assigned.

FIG. 8 is a scatter plot showing a correlation between baseline gene expression transformed RGE values for CXCL9 and TRIM29 in NSCLC patients from the trial. Clinical response status measured as best overall response (BOR) indicated by colors at (left) 6 weeks and (right) 12 weeks post dosing with anti-PD-L1. Red box indicates a group of PRs. SD=stable disease; PR=partial responder; PD=progressive disease; ND=no clinical response status assigned.

FIG. 9 is a scatter plot showing a correlation between percentage change in tumor size from baseline following treatment with MEDI4736 versus CXCL9 and IFNy mRNA in NSCLC squamous cell patient tumors. PR=partial responder; PD=progressive disease; SD=stable disease; NE=not evaluable.

FIG. 10 is a scatter plot between CXCL9 mRNA versus IFNγ mRNA in NSCLC squamous cell patient tumors. PR=partial responder; PD=progressive disease; SD=stable disease; NE=not evaluable.

FIG. 11 is a set of boxplots plots of CXCL9 mRNA within each response category in NSCLC squamous cell patient tumors. PR vs. PD p=0.06; PR vs. SD p=0.41; PR=partial responder; PD=progressive disease; SD=stable disease; NE=not evaluable.

FIG. 12 is a set of boxplots plots of IFNγ mRNA within each response category in NSCLC squamous cell patient tumors. PR vs. PD p=0.01; PR vs. SD p=0.43; PR=partial responder; PD=progressive disease; SD=stable disease; NE=not evaluable.

FIG. 13 is a scatter plot between percentage change in tumor size from baseline versus IFNγ mRNA in H&N squamous cell patient tumors. PR=partial responder; PD=progressive disease; SD=stable disease; NE=not evaluable.

FIG. 14 is a scatter plot between percentage change in tumor size from baseline versus CXCL9 mRNA in H&N squamous cell patient tumors. PR=partial responder; PD=progressive disease; SD=stable disease; NE=not evaluable.

FIG. 15 is a set of boxplots plots of IFNγ mRNA within each response category in H&N squamous cell patient tumors. PR vs. PD p=0.01; PR vs. SD p=0.43; PR=partial responder; PD=progressive disease; SD=stable disease; NE=not evaluable.

FIG. 16 is a set of boxplots plots of CXCL9 mRNA within each response category in H&N squamous cell patient tumors. PR vs. PD p=0.01; PR vs. SD p=0.43; PR=partial responder; PD=progressive disease; SD=stable disease; NE=not evaluable.

### LIST OF SEQUENCES

MEDI4736 light chain variable region amino acid sequence: SEQ ID NO:1

MEDI4736 heavy chain variable region amino acid sequence: SEQ ID NO:2.

MEDI4736 heavy chain variable region amino acid sequence of CDR1, CDR2, and CDR3: SEQ ID NOs:3-5.

MEDI4736 light chain variable region amino acid sequence of CDR1, CDR2, and CDR3: SEQ ID NOs:6-8.

### DETAILED DESCRIPTION OF THE INVENTION

The present invention provides methods for treating lung cancer (e.g., non-small cell lung cancer) with an anti-PD-L1 antibody in a patient identified using a polynucleotide or polypeptide marker of the invention (e.g., CXCL9, KRT8, TRIM29, and/or IFNgamma) In other embodiments, the present invention provides methods for treating lung cancer in a patient identified using any one or more of markers CXCL9, KRT8, TRIM29, and/or IFNgamma in combination with marker PD-L1.

The invention is based, at least in part, on the discovery that levels of CXCL9, KRT8, TRIM29, IFNgamma, and/or PD-L1 are differentially expressed in tumors (i.e., increased in a tumor sample) obtained from a subject suffering from lung cancer or head and neck cancer relative to a reference, and that this increased expression can be used to identify patients responsive to treatment with an anti-PD-L1 antibody. Accordingly, the invention provides methods for identifying subjects that have lung cancer that are likely to respond to anti-PD-L1 antibody treatment based on the level of CXCL9, KRT8, TRIM29, and/or IFNgamma, optionally in combination with marker PD-L1 expression, in a subject tumor sample.

### B7-H1/PD-L1

B7-H1, also known as PD-L1, is a type I transmembrane protein of approximately 53kDa in size. In humans B7-H1 is expressed on a number of immune cell types including activated and anergic/exhausted T cells, on naive and activated B cells, as well as on myeloid dendritic cells (DC), monocytes and mast cells. It is also expressed on non-immune cells including islets of the pancreas, Kupffer cells of the liver, vascular endothelium and selected epithelia, for example airway epithelia and renal tubule epithelia, where its expression is enhanced during inflammatory episodes. B7-H1 expression is also found at increased levels on a number of tumours including, but not limited to breast, colon, colorectal, lung, renal, including renal cell carcinoma, gastric, bladder, non-small cell lung cancer (NSCLC), hepatocellular cancer (HCC), and pancreatic cancer, as well as melanoma.

B7-H1 is known to bind two alternative ligands, the first of these, PD-1, is a 50-55 kDa type I transmembrane receptor that was originally identified in a T cell line undergoing activation-induced apoptosis. PD-1 is expressed on activated T cells, B cells, and monocytes, as well as other cells of the immune system and binds both B7-H1 (PD-L1) and the related B7-DC (PD-L2). The second is the B7 family member B7-1, which is expressed on activated T cells, B cells, monocytes and antigen presenting cells.

Signaling via the PD-1/B7-H1 axis is believed to serve important, non-redundant functions within the immune system, by negatively regulating T cell responses. B7-H1 expression on tumor cells is believed to aid tumors in evading detection and elimination by the immune system. B7-H1 functions in this respect via several alternative mechanisms including driving exhaustion and anergy of tumour infiltrating T lymphocytes, stimulating secretion of immune repressive cytokines into the tumour micro-environment, stimulating repressive regulatory T cell function and protecting B7-H1 expressing tumor cells from lysis by tumor cell specific cytotoxic T cells.

### Anti-PD-L1 Antibodies

Antibodies that specifically bind and inhibit PD-L1 activity (e.g., binding to PD-1 and/or CD80) are useful for the treatment of lung cancer (e.g., non-small cell lung cancer

MEDI4736 is an exemplary anti-PD-L1 antibody that is selective for B7-H1 and blocks the binding of B7-H1 to the PD-1 and CD80 receptors. MEDI4736 can relieve B7-H1-mediated suppression of human T-cell activation in vitro and inhibits tumor growth in a xenograft model via a T-cell dependent mechanism. Other agents that could be used include agents that inhibit PD-L1 and/or PD-1 (AB or other).

Information regarding MEDI4736 (or fragments thereof) for use in the methods provided herein can be found in International Application Publication No. WO 2011/066389 A1, the disclosure of which is incorporated herein by reference in its entirety. The fragment crystallizable (Fc) domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC).

MEDI4736 and antigen-binding fragments thereof for use in the methods provided herein comprises a heavy chain and a light chain or a heavy chain variable region and a light chain variable region. In a specific aspect, MEDI4736 or an antigen-binding fragment thereof for use in the methods provided herein comprises a light chain variable region comprising the amino acid sequence of SEQ ID NO:1 and a heavy chain variable region comprising the amino acid sequence of SEQ ID NO:2. In a specific aspect, MEDI4736 or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:3-5, and wherein the light chain variable region comprises the Kabat-defined CDR1, CDR2, and CDR3 sequences of SEQ ID NOs:6-8. Those of ordinary skill in the art would easily be able to identify Chothia-defined, Abm-defined or other CDR definitions known to those of ordinary skill in the art. In a specific aspect, MEDI4736 or an antigen-binding fragment thereof for use in the methods provided herein comprises the variable heavy chain and variable light chain CDR sequences of the 2.14H9OPT antibody as disclosed in U.S. Pat. No. 8,779,108; U.S. Patent Application Publication No. US2013034559; and in WO 2011/066389 A1, which are herein incorporated by reference in their entirety.

### Characterizing Responsiveness to Anti-PD-L1 Antibody Therapy

In characterizing the responsiveness of lung cancer in a subject to anti-PD-L1 antibody treatment, the level of CXCL9, KRT8, TRIM29, and/or IFNgamma expression, optionally in combination with PD-L1, is measured in different types of biologic samples (e.g., tumor sample).

CXCL9, KRT8, TRIM29, IFNgamma, and/or PD-L1 polynucleotide or polypeptide expression is higher in a tumor sample obtained from a subject that is responsive to anti-PD-L1 antibody treatment than the level of expression in a non-responsive subject (e.g., a subject with progressive disease). In one embodiment, an alteration in expression is calculated using cycle threshold (Ct) values. For example, the Ct value of a gene of interest (e.g., CXCL9, KRT8, TRIM29, IFNgamma, and/or PD-L1) is obtained and from that value the Ct value of a reference gene (e.g., B2M, ACTB, GAPDH) is subtracted from the mean Ct value for each gene to obtain a Delta-Ct value. The final gene expression score is defined as (20-ΔCt). In evaluating increased polypeptide expression (e.g., PD-L1, CXCL9, KRT8, TRIM29, IFNgamma), an immunohistochemical (IHC) score or IHC-membrane (IHC-M) score may be used. In other embodiments, expression of a marker of the invention is increased by at least about 2, 3, 4, 5 or 10-fold in a responsive patient relative to the level in a non-responsive subject (e.g., a subject with progressive disease). CXCL9, KRT8, TRIM29, IFNgamma, and/or PD-L1 polynucleotide or polypeptide fold change values are determined using any method known in the art, including but not limited to quantitative PCR, RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, colorimetric assays, mass spectrometry (e.g., laser desorption/ionization mass spectrometry), fluorescence (e.g. sandwich immunoassay), surface plasmon resonance, ellipsometry, and atomic force microscopy.

In particular embodiments, the responsiveness of lung cancer in a subject to anti-PD-L1 antibody treatment, is assayed using one of the following combinations of polynucleotide markers: CXCL9 and KRT8; CXCL9 and TRIM29; CXCL9 and IFNgamma; KRT8 and TRIM29; KRT8 and IFNgamma; TRIM29 and IFNgamma; CXCL9, KRT8, and TRIM29; KRT8, TRIM29, and IFNgamma; CXCL9, KRT8, TRIM29, and IFNgamma. PD-L1 may be added to any of the preceding groups of markers (e.g., CXCL9, KRT8, TRIM29, IFNgamma and PD-L1).

### Selection of a Treatment Method

Subjects suffering from lung cancer (e.g., non-small cell lung cancer) or head and neck cancer may be tested for CXCL9, KRT8, TRIM29, IFNgamma, and/or PD-L1 polynucleotide or polypeptide expression in the course of selecting a treatment method. Patients characterized as having high expression (e.g., as defined by Ct or IHC-M score) or increased expression relative to a reference level are identified as responsive to anti-PD-L1 treatment.

Treatment with an Anti-PD-L1 Antibody

Patients identified as having tumors that express CXCL9, KRT8, TRIM29, and/or IFNgamma, particularly at high levels, are likely to be responsive to anti-PD-L1 antibody therapy. Such patients are administered an anti-PD-L1 antibody, such as MEDI4736, or an antigen-binding fragment thereof. MEDI4736 or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

In some embodiments, at least two doses of MEDI4736 or an antigen-binding fragment thereof are administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, MEDI4736 or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, MEDI4736 or an antigen-binding fragment thereof is administered over a three-week treatment period, a six-week treatment period, over a nine-week treatment period, over a twelve-week treatment period, over a twenty-four-week treatment period, or over a one-year or more treatment period. In some embodiments, MEDI4736 or an antigen-binding fragment thereof is administered over a two-month treatment period, over a four-month treatment period, or over a six-month or more treatment period (e.g., during a maintenance phase).

The amount of MEDI4736 or an antigen-binding fragment thereof to be administered to the patient will depend on various parameters, such as the patient's age, weight, clinical assessment, tumor burden and/or other factors, including the judgment of the attending physician.

In certain aspects the patient is administered one or more doses of MEDI4736 or an antigen-binding fragment thereof wherein the dose is about 0.1 mg/kg. In certain aspects the patient is administered one or more doses of MEDI4736 or an antigen-binding fragment thereof wherein the dose is about 0.3 mg/kg. In certain aspects the patient is administered one or more doses of MEDI4736 or an antigen-binding fragment thereof wherein the dose is about 1 mg/kg.

In certain aspects the patient is administered one or more doses of MEDI4736 or an antigen-binding fragment thereof wherein the dose is about 3 mg/kg. In certain aspects the patient is administered one or more doses of MEDI4736 or an antigen-binding fragment thereof wherein the dose is about 10 mg/kg. In certain aspects the patient is administered one or more doses of MEDI4736 or an antigen-binding fragment thereof wherein the dose is about 15 mg/kg.

In certain aspects the patient is administered at least two doses of MEDI4736 or an antigen-binding fragment thereof wherein the dose is about 0.1 mg/kg. In certain aspects the patient is administered at least two doses of MEDI4736 or an antigen-binding fragment thereof wherein the dose is about 0.3 mg/kg. In certain aspects the patient is administered at least two doses of MEDI4736 or an antigen-binding fragment thereof wherein the dose is about 1 mg/kg. In certain aspects the patient is administered at least two doses of MEDI4736 or an antigen-binding fragment thereof wherein the dose is about 3 mg/kg. In certain aspects the patient is administered at least two doses of MEDI4736 or an antigen-binding fragment thereof wherein the dose is about 10 mg/kg. In certain aspects the patient is administered at least two doses of MEDI4736 or an antigen-binding fragment thereof wherein the dose is about 15 mg/kg. In some embodiments, the at least two doses are administered about two weeks apart. In some embodiments, the at least two doses are administered about three weeks apart.

In certain aspects the patient is administered at least three doses of MEDI4736 or an antigen-binding fragment thereof wherein the dose is about 0.1 mg/kg. In certain aspects the patient is administered at least three doses of MEDI4736 or an antigen-binding fragment thereof wherein the dose is about 0.3 mg/kg. In certain aspects the patient is administered at least three doses of MEDI4736 or an antigen-binding fragment thereof wherein the dose is about 1 mg/kg. In certain aspects the patient is administered at least three doses of MEDI4736 or an antigen-binding fragment thereof wherein the dose is about 3 mg/kg. In certain aspects the patient is administered at least three doses of MEDI4736 or an antigen-binding fragment thereof wherein the dose is about 10 mg/kg. In certain aspects the patient is administered at least three doses of MEDI4736 or an antigen-binding fragment thereof wherein the dose is about 15 mg/kg. In some embodiments, the at least three doses are administered about two weeks apart. In some embodiment, the at least three doses are administered about three weeks apart.

In certain aspects, administration of MEDI4736 or an antigen-binding fragment thereof according to the methods provided herein is through parenteral administration. For example, MEDI4736 or an antigen-binding fragment thereof can be administered by intravenous infusion or by subcutaneous injection. In some embodiments, the administration is by intravenous infusion.

In certain aspects, MEDI4736 or an antigen-binding fragment thereof is administered according to the methods provided herein in combination or in conjunction with additional cancer therapies. Such therapies include, without limitation, chemotherapeutic agents such as Vemurafenib, Erlotinib, Afatinib, Cetuximab, Carboplatin, Bevacizumab, Erlotinib, or Pemetrexed, or other chemotherapeutic agents, as well radiation or any other anti-cancer treatments.

The methods provided herein can decrease tumor size, retard tumor growth or maintain a steady state. In certain aspects the reduction in tumor size can be significant based on appropriate statistical analyses. A reduction in tumor size can be measured by comparison to the size of patient's tumor at baseline, against an expected tumor size, against an expected tumor size based on a large patient population, or against the tumor size of a control population. In certain aspects provided herein, the administration of MEDI4736 can reduce a tumor size by at least 25%. In certain aspects provided herein, the administration of MEDI4736 can reduce a tumor size by at least 25% within about 6 weeks of the first treatment. In certain aspects provided herein, the administration of MEDI4736 can reduce a tumor size by at least 50%. In certain aspects provided herein, the administration of MEDI4736 can reduce a tumor size by at least 50% within about 10 weeks of the first treatment. In certain aspects provided herein, the administration of MEDI4736 can reduce a tumor size by at least 75%. In certain aspects provided herein, the administration of MEDI4736 can reduce a tumor size by at least 75% within about 10 weeks of the first treatment.

In certain aspects, use of the methods provided herein, i.e., administration of MEDI4736 or an antigen-binding fragment thereof can decrease tumor size within 6 weeks, within 7 weeks, within 8 weeks, within 9 weeks, within 10 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks of the first treatment.

In some embodiments, administration of 1 mg/kg of MEDI4736 or an antigen-binding fragment thereof (e.g., at least one dose, at least two doses, at least three doses, at least four doses, at least five doses, at least six doses, at least seven doses, at least eight doses, at least nine doses, at least ten doses, or more every two weeks or every three weeks) can be sufficient to reduce tumor size. However, as provided herein, larger doses can also be administered, for example, to optimize efficacy, number of doses necessary, or certain pharmacokinetic parameters.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population.

In certain aspects, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≧50% relative to baseline. Confirmation can be obtained using a consecutive repeat assessment at least 4 weeks from the date of first documentation

“Progressive disease” (PD) refers to an increase in tumor burden ≧25% relative to the minimum recorded (nadir). Confirmation can be obtained by a consecutive repeat assessment at least 4 weeks from the date of first documentation. New, non-measurable lesions do not define PD.

“Stable disease” (SD) refers to not meeting the criteria for CR, PR, or PD.

In certain aspects, administration of MEDI4736 or an antigen-binding fragment thereof can increase progression-free survival (PFS).

In certain aspects, administration of MEDI4736 or an antigen-binding fragment thereof can increase overall survival (OS).

According to the methods provided herein, administration of MEDI4736 or an antigen-binding fragment thereof can result in desirable pharmacokinetic parameters. Total drug exposure can be estimated using the “area under the curve” (AUC). “AUC (tau)” refers to AUC until the end of the dosing period, whereas “AUC (inf)”refers to the AUC until infinite time. The administration can produce AUC (tau) of about 100 to about 2,500 d·μg/mL. The administration can produce a maximum observed concentration (Cmax) of about 15 to about 350 μg/mL. The half-life of the MEDI4736 or the antigen-binding fragment thereof can be about 5 to about 25 days. In addition, the clearance of the MEDI4736 or the antigen-binding fragment thereof can be about 1-10 ml/day/kg.

As provided herein, MEDI4736 or an antigen-binding fragment thereof can also decrease free B7-H1 levels. Free B7-H1 refers to B7-H1 that is not bound (e.g., by MEDI4736). In some embodiments, B7-H1 levels are reduced by at least 80%. In some embodiments, B7-H1 levels are reduced by at least 90%. In some embodiments, B7-H1 levels are reduced by at least 95%. In some embodiments, B7-H1 levels are reduced by at least 99%. In some embodiments, B7-H1 levels are eliminated following administration of MEDI4736 or an antigen-binding fragment thereof. In some embodiments, administration of MEDI4736 or an antigen-binding fragment thereof reduces the rate of increase of B7-H1 levels as compared, e.g., to the rate of increase of B7-H1 levels prior to the administration of MEDI4736 or an antigen-binding fragment thereof.

### Kits

The invention provides kits for characterizing the responsiveness of a subject to anti-PD-L1 antibody treatment. In one embodiment, the kit includes a therapeutic composition containing an effective amount of an antibody that specifically binds a PD-L1 polypeptide in unit dosage form.

A diagnostic kit of the invention provides a reagent (e.g., TaqMan primers/probes for CXCL9, KRT8, TRIM29, IFNgamma, and/or PD-L1 polynucleotide and housekeeping reference genes) for measuring relative expression of CXCL9, KRT8, TRIM29, IFNgamma, and/or PD-L1 polynucleotide. In other embodiments, the kit further includes reagents suitable for PD-L1 immunohistochemistry (e.g., anti-PD-L1 antibody).

In some embodiments, the kit comprises a sterile container which contains a therapeutic and/or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

In one embodiment, a kit of the invention comprises reagents for measuring CXCL9, KRT8, TRIM29, IFNgamma, and/or PD-L1 polynucleotide or polypeptide expression and a therapeutic anti-PD-L1 antibody. If desired, the kit further comprises instructions for measuring CXCL9, KRT8, TRIM29, IFNgamma, and/or PD-L1 polynucleotide or polypeptide expression and/or instructions for administering the anti-PD-L1 antibody to a subject having a lung cancer (e.g., non-small cell lung cancer, small cell lung cancer) selected as responsive to anti-PD-L1 antibody treatment. In particular embodiments, the instructions include at least one of the following: description of the therapeutic agent; dosage schedule and administration for treatment or prevention of lung cancer (e.g., non-small cell lung cancer, small cell lung cancer) or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### RNA Extraction and Real-time PCR analysis

Total RNA was extracted from snap-frozen tissue specimens collected from patients with non-small cell lung cancer (NSCLC) using the ZR RNA MicroPrep kit (Zymo Research, Orange, CA). RNA purity and concentration were determined spectrophotometrically (260/280>1.9). RNA quality was assessed on an Agilent 2100 Bioanalyzer using the RNA 6000 Nano LabChip®.

TaqMan Gene Expression assays were purchased from Applied Biosystems/Life Technologies. The assays include: CD274 (Assay ID: Hs01125301_ml); CXCL9 (Assay ID: Hs00171065_ml); IFNG (Assay ID: Hs00989291_ml); KRTS (Assay ID: Hs00361185_ml); KRT8 (Assay ID: Hs01595539_gl), and TRIM29 (Assay ID: Hs00232590_ml), as well as reference genes: ACTB (Hs01060665_gl), GAPDH (Assay ID: Hs02758991_gl) and B2M (Assay ID:Hs00187842_ml).

The BioMark™ Dynamic Array (Fluidigm, San Francisco, Calif.) microfluidics system allows for high throughput real-time PCR, producing high quality data with low variability. Single stranded cDNA was generated from 50 ng total RNA using the SuperScript® III First-Strand Synthesis SuperMix (Life Technologies). cDNA samples were pre-amplified using TaqMan Gene Expression Assays and TaqMan Pre-Amp Master Mix, according to the manufacturer's instructions. Reactions contained 5 μL of cDNA, 10 μL Pre-Amp Master Mix and 5 μL of 0.2X gene expression assay mix (comprised of all primer/probes to be assayed) for a final volume of 20 μL. Pre-amplified cDNA was assayed by Real-Time PCR with TaqMan Gene Expression Assays specific for target genes of interest and TaqMan Universal Master Mix (Life Technologies) using the BioMark™ instrument (Fluidigm).

To prepare samples for loading into 96×96 dynamic array chips (Fluidigm), the reaction mix contained 2.5μL 2X Universal Master Mix (Life Technologies), 0.25 μL Sample Loading Buffer (Fluidigm), and 2.25 μL pre-amplified cDNA. To prepare the primer/probes, the reaction mix contained 2.5 μL 20X TaqMan Gene Expression Assay and 2.5 μL Assay Loading Buffer (Fluidigm). Prior to loading the samples and assay reagents into the inlets, the chip was primed in the IFC Controller. Five microliters of sample prepared as described was loaded into each sample inlet of the dynamic array chip, and 5 μL of 10X gene expression assay mix was loaded into each detector inlet. Upon completion of the IFC priming and load/mixing steps, the chip was loaded on the BioMark™ Real-Time PCR System for thermal cycling.

Cycle threshold (Ct) values were generated using BioMark analysis software (Fluidigm Corporation). Ct values above 25 were excluded from calculations. Delta-Ct values were calculated by subtracting the mean Ct of 3 reference genes (B2M, ACTB, and GAPDH) from the mean Ct value for each gene evaluated. The final gene expression score (see the figures) is defined as (20-ΔCt), so that this score is positive overall and high score represents high expression in the log2 scale.

### Example 2

### Strong PD-L1 Expression Correlates with a Subject's Responsiveness to Anti-PD-L1 Antibody Treatment

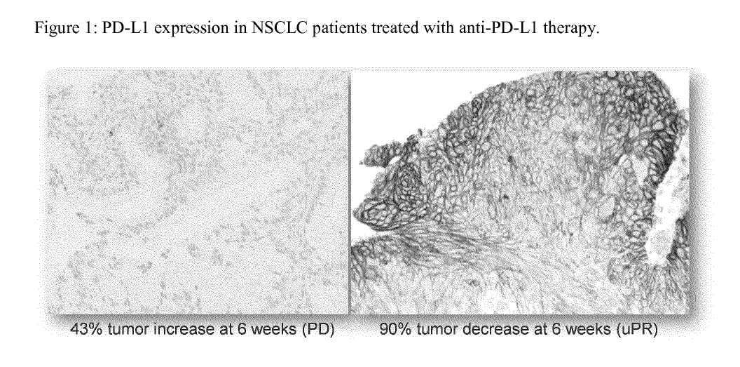

PD-L1 membrane expression can be measured using immunohistochemistry (FIG. 1). Non-small cell lung cancer (NSCLC) patients with strong PD-L1 membrane expression typically respond to anti-PD-L1 antibody treatment. Whereas patients having little or undetectable levels of PD-L1 membrane expression are less responsive to anti-PD-L1 antibody treatment.

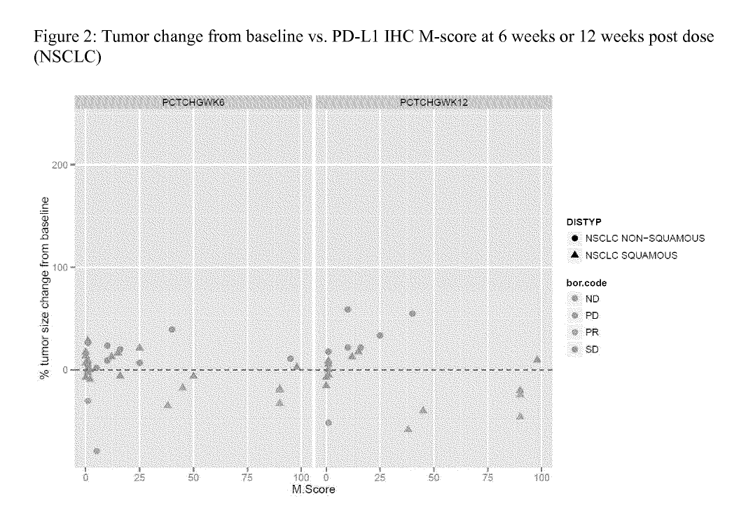

Immunohistochemical (IHC) results of PD-L1 membrane (M) expression can be expressed numerically as an IHC-M score. FIG. 2 shows the correlation between percent tumor size change from baseline and IHC M-score of PD-L1. The IHC M score is defined as the percentage of tumor cells with PD-L1 staining in NSCLC patients from the trial. Clinical response status measured as best overall response (BOR) indicated by colors at (left) 6 weeks and (right) 12 weeks post treatment with anti-PD-L1.

### Example 3

### PD-L1 Antibody Therapy Alters Gene Expression

A real time gene express assay (i.e., TaqMan assay) was used to determine how anti-PD-L1 antibody therapy altered the expression of candidate genes in patients treated with an anti-PD-L1 antibody. More specifically, the expression of T cell subtype transcripts, cytokine/chemokine transcripts, known IMT (immune modulatory therapy) transcripts, NSCLC subtype transcripts, and other immune-specific transcripts was measured. A complete list of assayed gene is provided below.

In the heatmap, genes that are highly expressed are indicated in red. Genes with low expression are indicated in blue. Highly expressed genes CXCL9, KRT8, TRIM29, and IFNgamma were selected for further analysis.

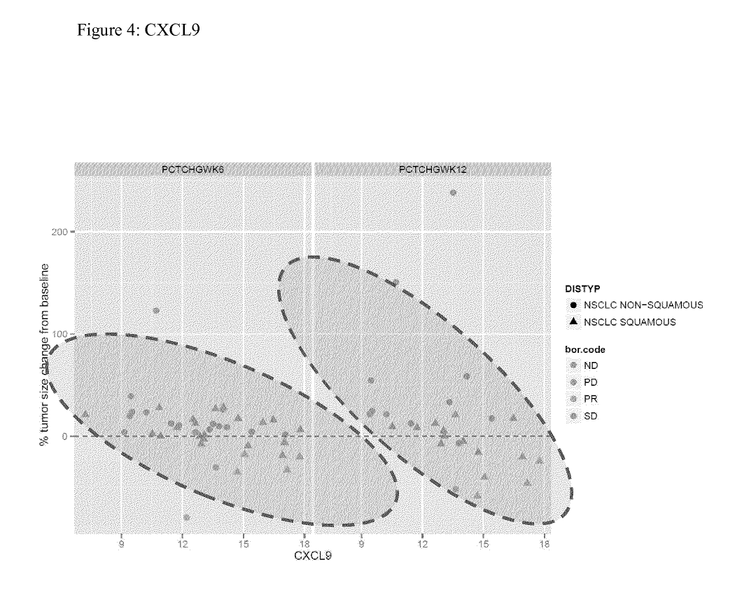

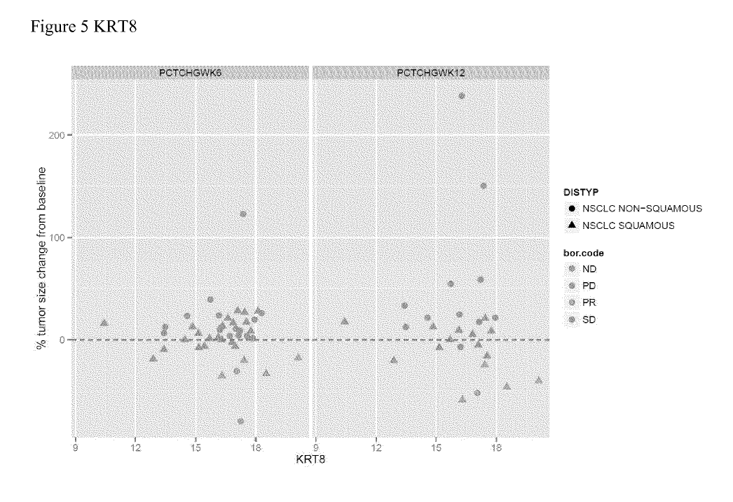

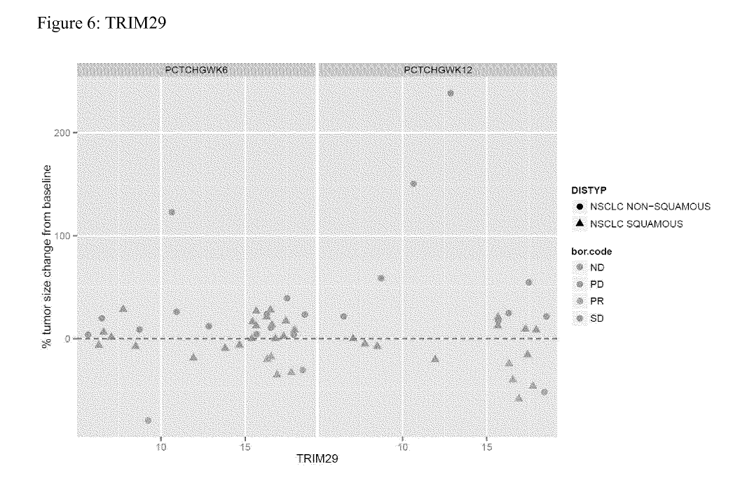

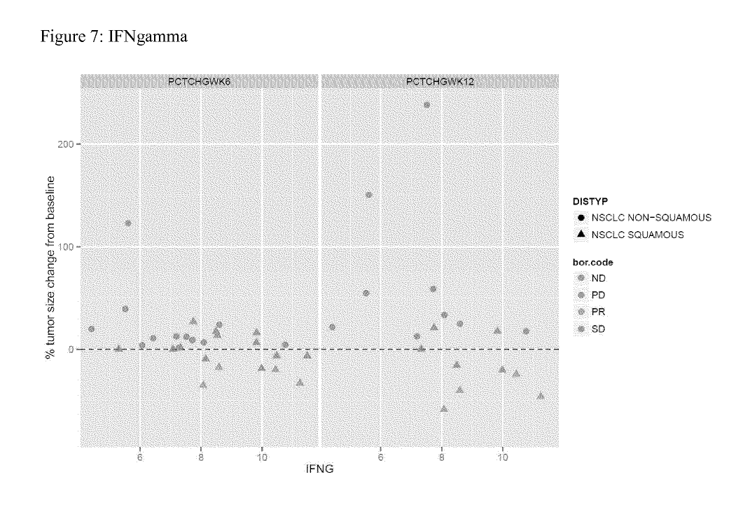

FIGS. 4, 5, 6, and 7 show results of an analysis of tumor size change relative to CXCL9, KRT8, TRIM29, and IFNgamma score, respectively. As indicated, each of CXCL9, KRT8, TRIM29, and IFNgamma are useful markers for identifying patients that are responsive to treatment with an anti-PD-L1 antibody.

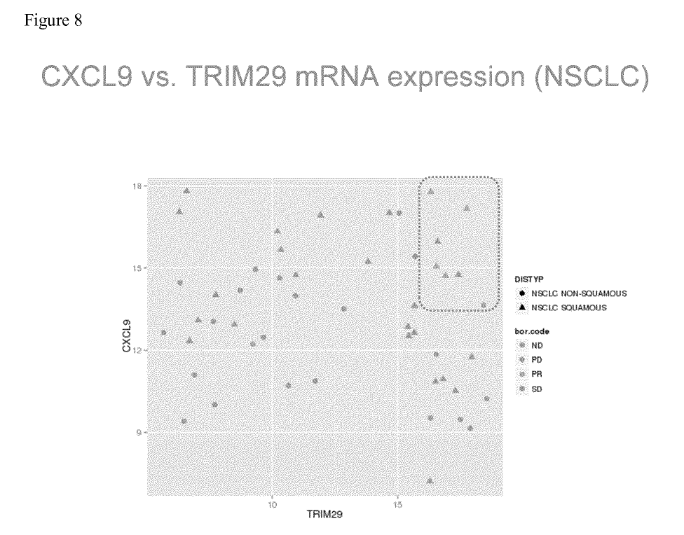

FIG. 8 shows results with CXCL9 score relative to TRIM29. Subjects that were particularly responsive to treatment with anti-PD-L1 antibody had tumors showing high expression of CXCL9 and TRIM29.

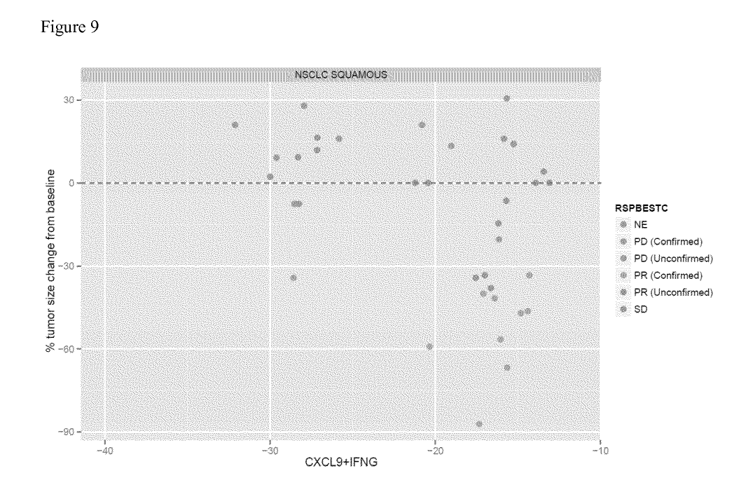

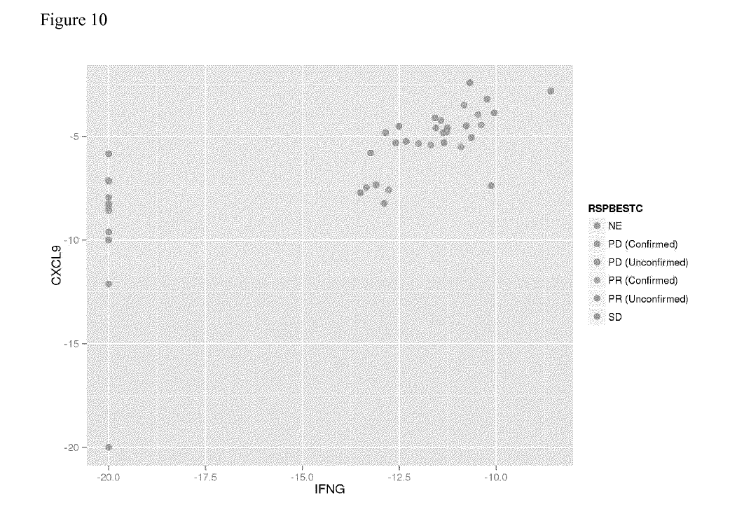

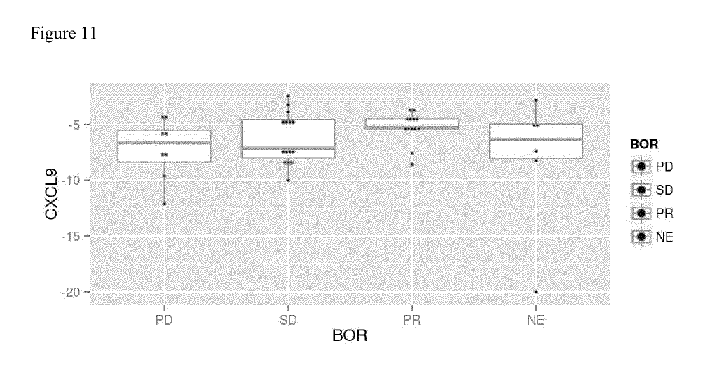

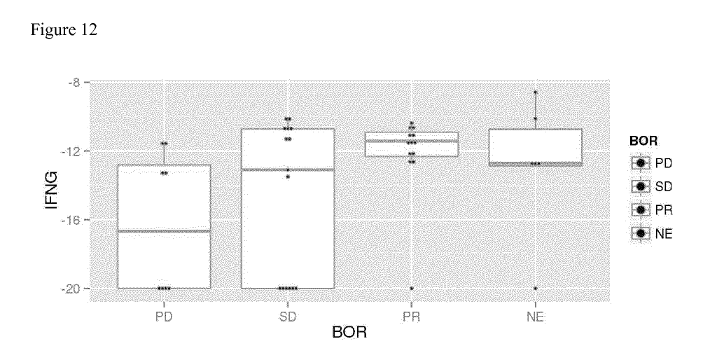

Expression of CXCL9 and INFgamma correlates strongly with response to anti-PD-L1 antibody therapy by showing significant reductions in NSCLC tumor size versus baseline (FIGS. 9) and response (FIG. 10). The correlation between the response to anti-PD-L1 treatment and expression of CXCL9 (FIG. 11) and IFNgamma (FIG. 12) was also demonstrated, where patients with partial response or stable disease upon treatment had tumors that expressed higher levels of these markers.

### Example 4

### Head and Neck Cancer Response to Anti-PD-L1 Therapy Correlates with Gene Expression

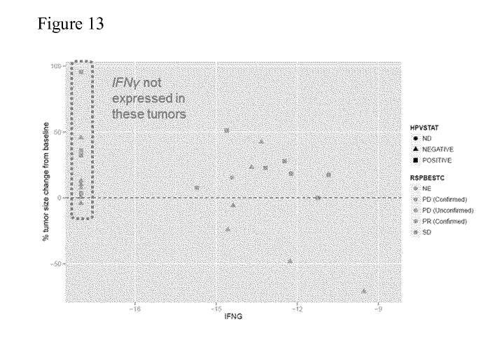

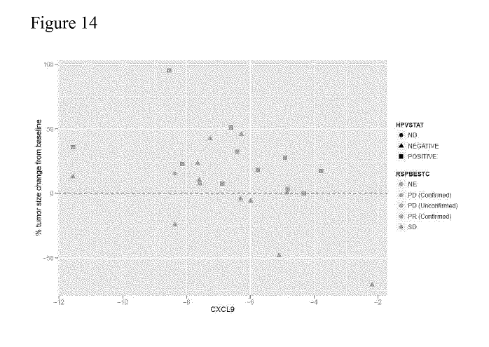

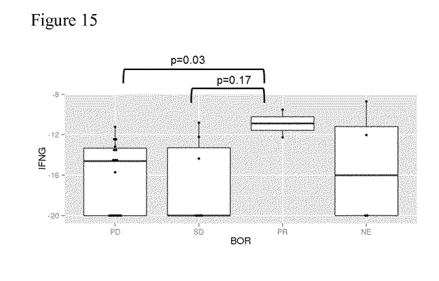

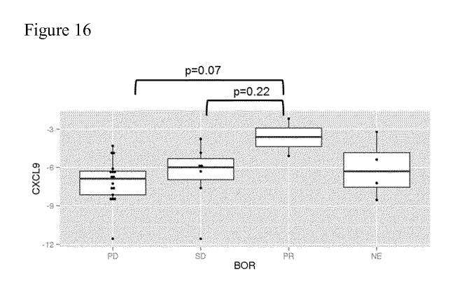

The relationship between gene expression patterns and tumor response to anti-PD-L1 therapy was investigated. Tumor samples from Head and Neck cancer patients undergoing anti-PD-L1 therapy (MEDI4736) were collected and gene expression patterns were analyzed. As shown in FIGS. 13 and 14, expression of IFNgamma (FIG. 13) and CXCL9 (FIG. 14) correlated with response rate and percentage tumor size change in response to anti-PD-L1 (MEDI4736) treatment. The correlation between the response to anti-PD-L1 treatment and expression of IFNgamma (FIG. 15) and CXCL9 (FIG. 16) was also demonstrated, where patients with partial response or stable disease upon treatment had tumors that expressed higher levels of these markers. These data demonstrate that IFNgamma and/or CXCL9 expression can be used to identify Head and Neck cancer patients that are more likely to benefit from anti-PD-L1 therapy. These markers can also, therefore, be used to increase the efficacy of treatment by selectively targeting patients having tumors that express these markers.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

SEQUENCE LISTING

> PCT/US2010/058007_77 Sequence 77 from
PCT/US2010/058007 Organism: Homo sapiens
SEQ ID NO: 1
EIVLTQSPGTLSLSPGERATLSCRASQRVSSSYLAWYQQKPGQAPRLL
IYDASSRATGIPDRFSGSGSGTDFTLTISRLEPEDFAVYYCQQYGSLP
WTFGQGTKVEIK
> PCT/US2010/058007_72 Sequence 72 from
PCT/US2010/058007 Organism: Homo sapiens
SEQ ID NO: 2
EVQLVESGGGLVQPGGSLRLSCAASGFTFSRYWMSWVRQAPGKGLEWV
ANIKQDGSEKYYVDSVKGRFTISRDNAKNSLYLQMNSLRAEDTAVYYC
AREGGWFGELAFDYWGQGTLVTVSS
VH CDR1
> PCT/US2010/058007_73 Sequence 73 from
PCT/US2010/058007 Organism: Homo sapiens
SEQ ID NO: 3
RYWMS
VH CDR2
> PCT/US2010/058007_74 Sequence 74 from
PCT/US2010/058007 Organism: Homo sapiens
SEQ ID NO: 4
NIKQDGSEKYYVDSVKG
VH CDR3
> PCT/US2010/058007_75 Sequence 75 from
PCT/US2010/058007 Organism: Homo sapiens
SEQ ID NO: 5
EGGWFGELAFDY
VL CDR1
> PCT/US2010/058007_78 Sequence 78 from
PCT/US2010/058007 Organism: Homo sapiens
SEQ ID NO: 6
RASQRVSSSYLA
VL CDR2
> PCT/US2010/058007_79 Sequence 79 from
PCT/US2010/058007 Organism: Homo sapiens
SEQ ID NO: 7
DASSRAT
VL CDR3
> PCT/US2010/058007_80 Sequence 80 from
PCT/US2010/058007 Organism: Homo sapiens
SEQ ID NO: 8
QQYGSLPWT

What is claimed is:

1. A method of treatment comprising administering an anti-PD-L1 antibody, or an antigen binding fragment thereof, to a patient identified as having a non-small cell lung cancer or a head and neck tumor that expresses one or more markers selected from the group consisting of CXCL9, KRT8, TRIM29, and IFNgamma.

2. The method of claim 1, wherein the anti-PD-L1 antibody is MEDI4736.

3. A method of treatment comprising administering MEDI4736 or an antigen binding fragment thereof to a patient identified as having a non-small cell lung cancer or a head and neck tumor that expresses one or more markers selected from the group consisting of CXCL9, KRT8, TRIM29, and IFNgamma.

4. The method of any of claims 1-3, wherein the patient is identified as responsive to MEDI4736.

5. The method of any of claims 1-4, wherein the patient is further identified as having a tumor expressing PD-L1.

6. The method of any of claims 1-5, wherein the tumor expresses CXCL9 and IFNgamma.

7. The method of any of claims 1-5, wherein the tumor expresses CXCL9 and KRT8.

8. The method of any of claims 1-5, wherein the tumor expresses CXCL9 and TRIM29.

9. The method of any of claims 1-5, wherein the tumor expresses CXCL9, KRT8, TRIM29, and IFNgamma.

10. The method of any of claims 1-5, wherein the tumor expresses CXCL9, KRT8, TRIM29, IFNgamma, and PD-L1.

11. The method of any of claims 1-10, wherein marker gene expression is detected in a Real-Time PCR assay.

12. The method of any of claims 1-10, wherein marker polypeptide expression is detected using immunohistochemistry.

13. The method of claim 12, wherein the tumor cells are formalin fixed and paraffin embedded.

14. The method of any of claims 1-10, wherein the non-small cell lung cancer is selected from the group consisting of squamous cell carcinoma, non-squamous cell carcinoma, adenocarcinoma, large cell carcinoma, adenosquamous carcinoma and sarcomatoid carcinoma.

15. The method of any of claims 1-10, wherein at least about 0.1, about 0.3, about 1, about 3, about 10, or about 15 mg/kg MEDI4736, or an antigen-binding fragment thereof, is administered

16. The method of claim 15, wherein about 1 mg/kg MEDI4736, or an antigen-binding fragment thereof, is administered.

17. The method of claim 16, wherein about 3 mg/kg MEDI4736, or an antigen-binding fragment thereof, is administered.

18. The method of claim 17, wherein about 10 mg/kg MEDI4736 or an antigen-binding fragment thereof is administered.

19. The method of claim 18, wherein about 15 mg/kg MEDI4736, or an antigen-binding fragment, thereof is administered.

20. The method of any of claims 1-10, wherein the administration is repeated about every 14 or 21 days.

21. The method of any of claims 1-10, wherein at least two doses is administered.

22. The method of any of claims 1-10, wherein at least three doses is administered.

23. The method of any of claims 1-10, wherein at least five doses is administered.

24. A method of identifying a subject having non-small cell lung cancer or head and neck cancer responsive to anti-PD-L1 therapy, the method comprising detecting an increase in the level of one or more markers selected from the group consisting of CXCL9, KRT8, TRIM29, and IFNgamma in a non-small cell lung cancer or head and neck tumor of the subject, relative to a reference, thereby identifying said non-small cell lung cancer or head and neck cancer as responsive to anti-PD-L1 therapy.

25. The method of claim 24, further comprising detecting PD-L1 expression in the tumor.

26. The method of any one of claims 1-24, wherein the markers are detected by a method selected from the group consisting of real-time PCR, DNA microarray, immunostaining, ELISA, FACS, radioimmunoassay, immunoblot, Western blot, immunofluorescence, and immunoprecipitation.

27. A set of primers and/or probes for characterizing non-small cell lung cancer (NSCLC) or head and neck cancer wherein the primers and/or probes hybridize to two or more polynucleotide markers selected from the group consisting of CXCL9, KRT8, TRIM29, and IFNgamma.

28. A set of primers and/or probes for characterizing non-small cell lung cancer (NSCLC) wherein the primers and/or probes hybridize to three or more polynucleotide markers selected from the group consisting of CXCL9, KRT8, TRIM29, and IFNgamma.

29. The set of primers and/or probes of claim 27 or 28, comprising markers CXCL9, KRT8, TRIM29, and IFNgamma.

30. A kit comprising the primers and/or probes of any one of claim 27 or 28.

31. The kit of claim 30, further comprising a reagent to measure the level of PD-L1.

32. A method of increasing the efficacy of anti-PD-L1 therapeutic antibody treatment in a lung cancer or head and neck cancer patient comprising administering anti-PD-L1 therapeutic antibody to a lung cancer patient or a head and neck cancer patient identified as expressing a marker selected from CXCL9, KRT8, TRIM29, and IFNgamma.


SEQUENCE LISTING (14 sequences)

SEQ ID NO: 1 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 11 aa, Homo sapiens
GFTFSFRYWMS

SEQ ID NO: 4 - protein, 17 aa, Homo sapiens
NIKQDGSEKYYVDSVKG

SEQ ID NO: 5 - protein, 12 aa, Homo sapiens
EGGWFGELAFDY

SEQ ID NO: 6 - protein, 12 aa, Homo sapiens
RASQRVSSSYLA

SEQ ID NO: 7 - protein, 7 aa, Homo sapiens
DASSRAT

SEQ ID NO: 8 - protein, 9 aa, Homo sapiens
QQYGSLPWT

SEQ ID NO: 9 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 10 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 11 - protein, 588 aa, Homo sapiens
MEAADASRSNGSSPEARDARSPSGPSGSLENGTKADGKDAKTTNGHGGEAAEGKSLGSALKPGEGRSALFAGNEWRRPIIQFVESGDDKNSNYFSMDSMEGKRSPYAGLQLGAAKKPPVTFAEKGELRKSIFSESRKPTVSIMEPGETRRNSYPRADTGLFSRSKSGSEEVLCDSCIGNKQKAVKSCLVCQASFCELHLKPHLEGAAFRDHQLLEPIRDFEARKCPVHGKTMELFCQTDQTCICYLCMFQEHKNHSTVTVEEAKAEKETELSLQKEQLQLKIIEIEDEAEKWQKEKDRIKSFTTNEKAILEQNFRDLVRDLEKQKEEVRAALEQREQDAVDQVKVIMDALDERAKVLHEDKQTREQLHSISDSVLFLQEFGALMSNYSLPPPLPTYHVLLEGEGLGQSLGNFKDDLLNVCMRHVEKMCKADLSRNFIERNHMENGGDHRYVNNYTNSFGGEWSAPDTMKRYSMYLTPKGGVRTSYQPSSPGRFTKETTQKNFNNLYGTKGNYTSRVWEYSSSIQNSDNDLPVVQGSSSFSLKGYPSLMRSQSPKAQPQTWKSGKQTMLSHYRPFYVNKGNGIGSNEAP

SEQ ID NO: 12 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 13 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 14 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa